(19) United States
(12) Patent Application Publication
(10) Pub. No.: US 2022/0226517 A1
(43) Pub. Date: Jul. 21, 2022

(54) Location Oriented Monitoring and Sterilization System

(71) Applicant: Andrew Bennett, Belmont, MA (US)

(72) Inventor: Andrew Bennett, Belmont, MA (US)

(21) Appl. No.: 17/151,605

(22) Filed: Jan. 18, 2021

(51) Int. Cl. A61L 2/10; A61L 2/24; A61L 2/28

(52) U.S. Cl. CPC A61L 2/10; A61L 2/24; A61L 2/28; A61L 2202/11; A61L 2202/16; A61L 2202/14; A61L 2202/25

(57) ABSTRACT

The present invention relates to a system for and method of monitoring and sterilizing an enclosed space. A particular aspect of the invention is the use of an ultraviolet light producer. The system is controlled by and the process incorporates the use of computing technology. In summary, the invention sterilizes the applicable enclosed space by (A) monitoring the enclosed space based upon the information supplied by the computing technology, (B) sending a signal to the ultraviolet light producer to maximize the ultraviolet illumination disinfecting, and (C) using a model of the enclosed space to guide the positioning of the system throughout the disinfecting process. A final element of a general embodiment of the system would preferably be a communication element that can transmit data about the positioning of the system, and other pertinent information, to a remote device.

### BACKGROUND

These teachings relate generally to systems for and methods of sterilizing an enclosed space with ultraviolet light using with information about the characteristics and contents of the enclosed space and while level of sterilization is monitored during the process.

### BRIEF SUMMARY

The present invention, in the form of a system, includes, in a more general form, the elements for monitoring and sterilizing an enclosed space. The aforementioned elements in a housing for the system. This housing would be preferably sized and configured to contain the other elements of the system that would need to be within the enclosed space for the optimal performance of the system. One of such elements would be a power source situated within the housing. Another element would be an ultraviolet light producer, situated within and exposed on the outer surface of the housing and electronically connected to the power source. The power source would preferably be controlled (on and off) by a power switch, situated in association with the housing and electronically connected to the power source. Also within the housing would be computing technology, also connected to the power source. This computing technology would preferably (A) monitor the location of the system through the information supplied by the computing technology, (B) send a signal to the ultraviolet light producer to maximize the ultraviolet illumination disinfecting the enclosed space to the maximum degree allowable based upon the anticipated levels within, and areas of, the enclosed space need sterilization, and (C) use a model of the enclosed space to provide guidance regarding the desired positioning of the system throughout the disinfecting process. To facilitate achievement of the desired level of sterilization, a location detector for detecting the position of the system within the enclosed space could also be utilized. This locator would preferably be electronically connected to the computing technology. A final element of a general embodiment of the system would preferably be a communication element. Preferably the communication element would be situated within the housing and connected to both the power source and the computing technology. It would be preferred that the communication element be used to transmit data about the positioning of the system, and other pertinent information, to a remote device.

The present invention, as a method, includes, in a more general form, the steps for monitoring and sterilizing an enclosed space. It is essential in the execution of the inventive process to have information about the characteristics and contents of the enclosed space to be sterilized. Accordingly, one of the steps of the method is the modeling the three-dimensional configuration of the enclosed space. Once the modeling is completed (or alternatively while it is underway, depending upon the embodiment of the present invention), the modeling information is preferably fed into computing technology. The computed modeling information and the anticipated sterilization-level information are used to cause an ultraviolet light producing element to sterilize the enclosed space. Through the foregoing is the computed information dictates the production of the ultraviolet light and the ultraviolet light producing element can be positioned in a fashion that maximizes the ultraviolet illumination to disinfect the enclosed space to the maximum degree allowable based upon the levels and areas of sterilization needed. Through the illumination, there is monitoring of the location of the ultraviolet producing element relative to the areas of the enclosed space in need of sterilization.

### BRIEF DESCRIPTION OF THE FIGURES

FIG. 1 shows an embodiment of the present inventive system with a cutaway depicting a partial layout representation of the enclosed space in which the system could be used.

FIG. 2 shows an example of a schematic of the electronic elements of present inventive system.

FIG. 3 shows a version of the present invention focusing on location detection components.

FIG. 4 shows an embodiment the present invention focusing on sterilization-level detection.

FIG. 5 shows an enhanced version of the present invention, with a combination of the elements in the prior figures.

FIG. 6 shows the screen of a mobile device, like a smartphone, through which an operator of the present inventive system can see views of the placement of the system.

FIG. 7 shows a flowchart of a process version of the present invention through which an enclosed space can be sterilized,

FIG. 8 shows a flow chart depicting the method of FIG. 7 with additional step of detecting and monitoring the location of the ultraviolet producing element.

FIG. 9 shows a third flow chart, a different embodiment of the present method, with an additional step of storing data.

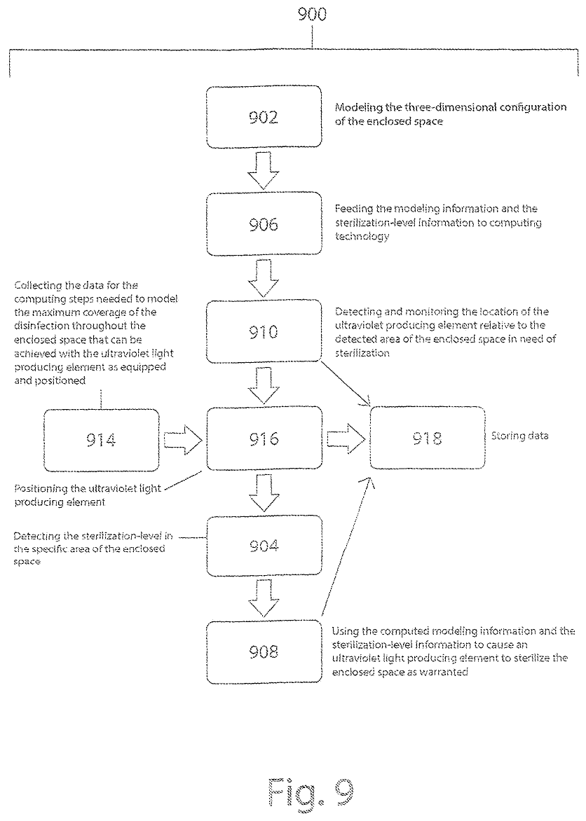

### DETAILED DESCRIPTION OF THE EMBODIMENTS OF THE PRESENT INVENTION

For the convenience of the read, Attachment A is a list of all of the elements of the system and steps of the method shown in the Figures.

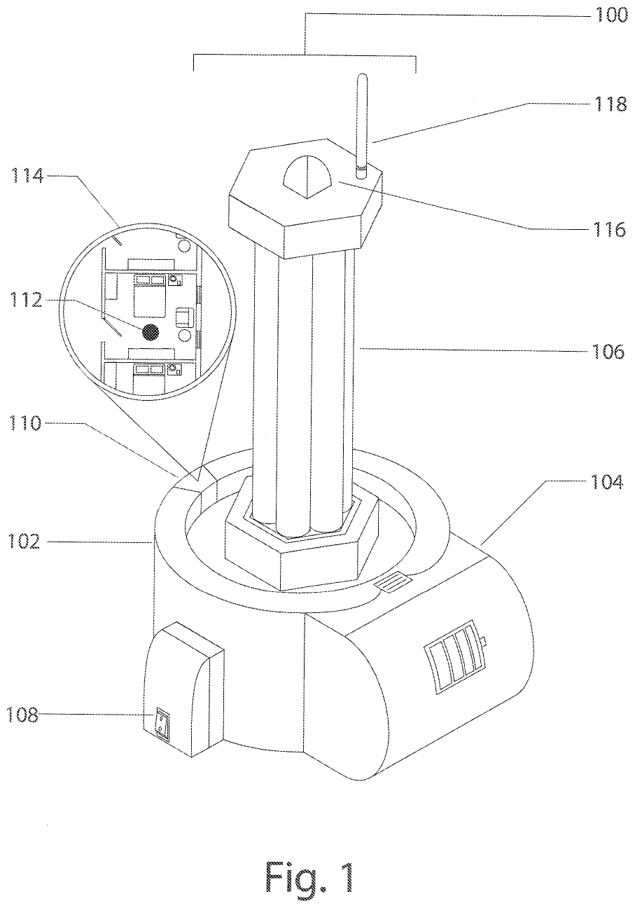

FIG. 1 shows an embodiment of the present monitoring and sterilizing invention in its broadest sense and situated in enclosed space 114 (shown in as cutaway—with system 100 position 112 indicating a possible placement of the invention). System 100 includes housing 102. In this particular embodiment, communication transmitter/receiver 118, along with location detector 116, is mounted on the top of housing 102. Also mounted on top of housing 102 is ultraviolet light producing element 106. Mounted on the walls of enclosed space 114, in this example, could be cameras, radars or other spatial sensors, each with a signal transmitter. Internal to housing 102, not explicitly shown in FIG. 1 but shown in subsequent figures and discussed in more detail herein below, are computing technology 110 and power source 104. Power source 104 activates the invention through the toggling on/off of power switch 108.

In this embodiment, the wall-mounted cameras or other spatial sensors could be capable of detecting the location of housing 102 within enclosed space 114. The detected position of housing 102. System position 112 in this example, can be communicated to housing 102 by signals transmitted from the cameras or sensors to communication transmitter/receiver 118. The positioning information provided through signals are used with other information available to computing technology 110 to affect the output of ultraviolet light producing element 106. Further, depending upon the configuration, communication transmitter/receiver 118 could be used to transmit data, to a remote device, about, for example, the positioning of housing 102, the levels of disinfecting within the specific areas of enclosed space 114 (possibly as reflected by the intensity and time of ultraviolet emission), and other pertinent information.

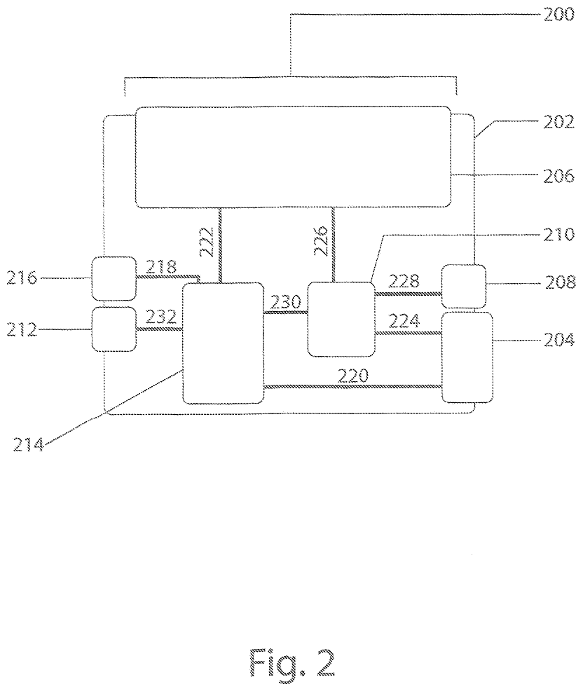

FIG. 2 shows an example of a schematic of the electronic elements of system 100 [now referred to as system 200]. In this embodiment, housing 202 has exposed to its external environment both communication transmitter/receiver 204 and ultraviolet light producing element 206. Internal and exposed to the external environment of housing 202 only (in this particular embodiment) via data port 208, is computing technology 210. Similar, within housing 202, and exposed to the external environment of housing 202 only (in this particular embodiment) via power port 212 and connected through electrical wire 232, is power source 214. Power source 214 is turned on/off by switch 216, which is exposed on the outer surface of housing 202 and is electronically connected to power source 214 by electrical wire 218 and to computing technology 210 by electrical wire 230. Similar, in this particular embodiment, power source 214 is electronically connected to communication transmitter/receiver 204 (through electrical wire 220), to ultraviolet light producing element 206 (through electrical wire 222), and to computing technology 210. In turn, computing technology 210 is electronically connected to communication transmitter/receiver 204 (through electrical wire 224), to ultraviolet light producing element 206 (through electrical wire 226) and to data port 208 (through electrical wire 228).

In the FIG. 2 configuration, computing technology 210 is cable of monitoring the location of housing 202 through the information supplied to computing technology 210 via communication transmitter/receiver 204 (e.g., from signals similar to camera signals discussed in relation to the FIG. 1 embodiment) and/or through information uploaded into computing technology 210 via data port 208 (e.g., certain details of the configuration of the enclosed space). By way of elaboration, computing technology 210 is capable of using a model of the enclosed space to provide guidance regarding the desired positioning of housing 202 throughout the disinfecting process. With that information, computing technology 210 is capable of sending a signal to ultraviolet light producing element 206 to maximize the ultraviolet illumination disinfecting the enclosed space to the maximum degree allowable based upon the anticipated levels within, and areas of, the enclosed space needing sterilization.

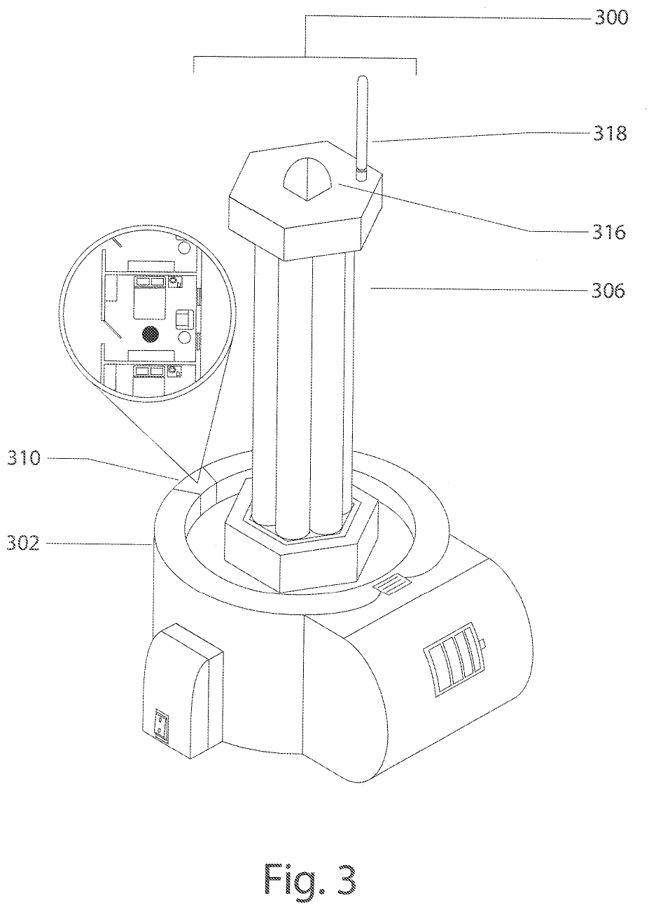

FIG. 3 shows a version of the embodiment shown the schematic of FIG. 2 (as system 300 in FIG. 3), with focus on location detector 316. In this particular embodiment, location detector 316 is electronically connected to computing technology 310, positioned within housing 302. System 300 of this embodiment is more inclusive and autonomous than system 100 of FIG. 1 in that the function of wall-mounted cameras or spatial sensors of system 100 are handled by location detector 316 of system 300. In this case, location detector 316 could be, for example, GPS-based technology that has enough precision to discern the position of system 300 with an enclosed space. One of ordinary skill in the art would realize other position detecting technology could also be used in the placement of system 300 as desired. Communications transmitter/receiver 318 and ultraviolet light producing element 306 are comparable for the similarly presented elements in FIGS. 1 and 2.

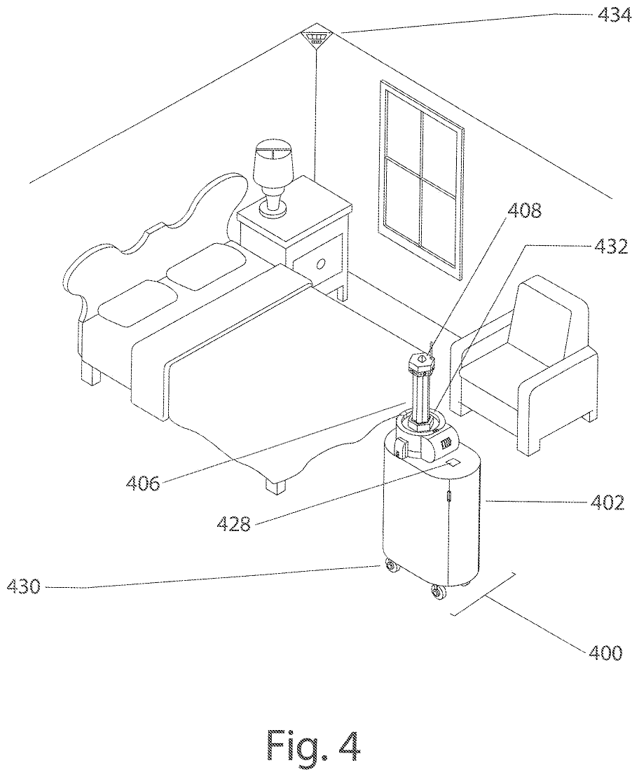

FIG. 4 shows the embodiment shown in FIG. 3, inclusive of system 300 (now identified as system 400), with sterilization-level detector 428 within and exposed to the outer surface of housing 402. In this embodiment of the present invention, based in part upon the location information fed to the computing technology by location detector 408, and the information fed to the computing technology by sterilization-level detector 428 (also electronically connected to the computing technology), the computing technology sends a signal to ultraviolet light producing element 406 for emission of the ultraviolet light within the enclosed space. Sterilization-level detector 428 is capable of detecting the levels of sterilization within the areas of the enclosed space that need sterilization, and provides the detail the computing technology needs to model the maximum coverage of the disinfection throughout the enclosed space that can be achieved with ultraviolet light producing element 406 as equipped and positioned. As such, the location and sterilization-level information are used to maximize the ultraviolet illumination disinfecting the enclosed space to the maximum degree allowable based upon the infected levels within and upon the areas of the enclosed space detected as in need of being sterilized. Further, using the information captured by system 400, the computing technology therein can cause propulsion element 430 to move system 400 to the locations from which system 300 can produce ultraviolet light for the best coverage and exposure for the enclosed space. Such information would be inclusive of data from attached mapping technology 432 and external supplement mapping source 434, the latter of which could be, for example, a camera or other position capturing/sensing device.

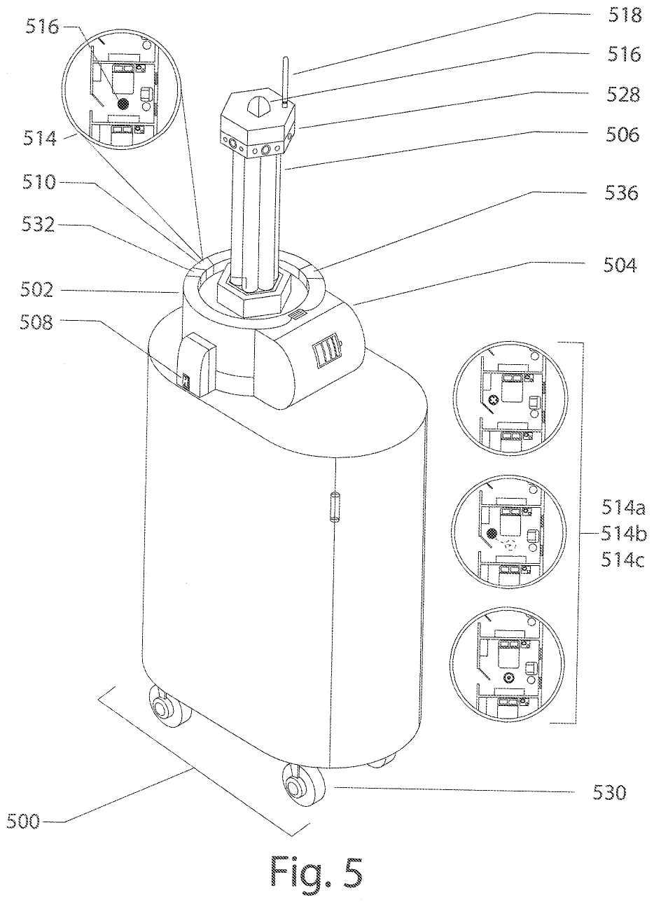

In a further embodiment of the inventive system—as shown FIG. 5, system 500 comprises elements similar to system 400 shown in FIG. 4. Propulsion element 530 is attached to housing 502 and connected to power source 504 and computing technology 510. System 500 can be positioned, through the activation of propulsion element 530, based in part upon the information received by computing technology 510 to facilitate the maximum number and levels of sterilized portions of an enclosed space. In this more enhanced embodiment of the present invention, system 500 also includes attached mapping technology 532. Attached mapping technology 532 could be housed within housing 502. Preferably, attached mapping technology 532 is connected to power source 504 and computing technology 510. With these connections, attached mapping technology 532 enables computing technology 510 to receive information about obstacles and other physical factors about the enclosed space. With this information, system 500 can be positioned to maximize the ultraviolet illumination in disinfecting the enclosed space with reference to the mapped obstacles and other physical factors. Further, both the mapping and the positioning of ultraviolent light producing element 506 during illumination can be in three dimensions.

Also shown in FIG. 5 is data storage 536. In a preferred configuration, data storage 536 is situated within housing 502 and connected to both power source 504 and computing technology 510. The stored data could include, for example, data about the positioning of system 500, the levels of disinfecting within the specific areas of the enclosed space, and other pertinent information. Beyond the foregoing, data about the relative position of system 500 in relation to obstacles detected in the enclosed space, the desired position of system 500 in relation to obstacles detected in enclosed space 514, or a combination of the foregoing (and other information) could be displayable. With the availability of communication element 518, the actual and optimal coverage of system 500 could be communicated to system 500 and stored in data storage 536 such that the relative level of disinfection of all parts of the enclosed space can be determined and completed.

In addition to some of the elements shown in earlier figures (notably, power switch 508, sterilization-level detector 528, and location detector 516), FIG. 5 also shows the positioning of system 500 in its operation. Device position 516, in this example depiction of enclosed space 514, is an illustration of an optimal placement of system 500. Views of enclosed space 514a, 514b and 514c show how propulsion element 530, powered by power source 504 and guided by computer technology 510 with information from sterilization-level detector 528, location detector 516, and attached mapping technology 532, can cause system 500 to be moved into the best position within enclosed space 514. Thus, through a combination of the mapping technology (to avoid obstacles) and the sterilization-level detectors (to determine blind spots), system 500 can navigate to a location of optimal coverage.

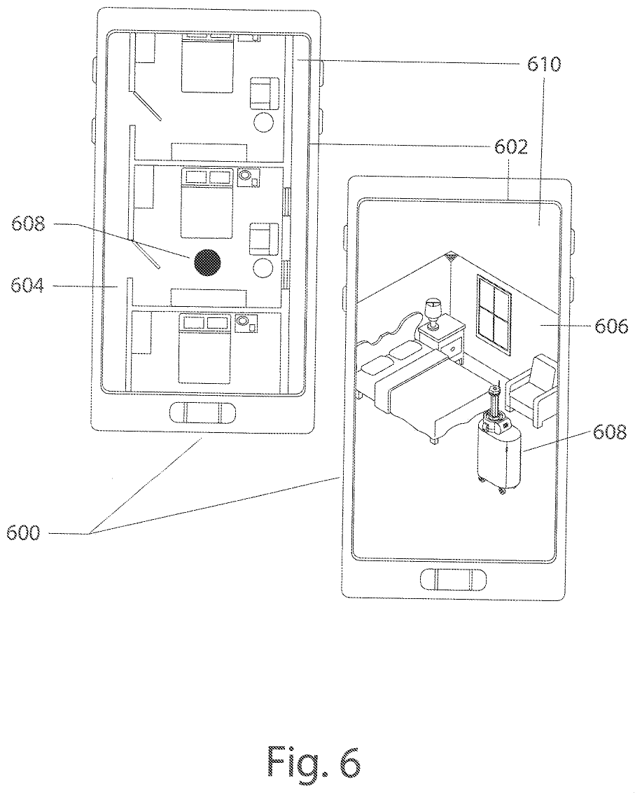

FIG. 6 shows the screen of mobile device 600, like a smartphone, through which an operator of the present inventive system can see views of the placement of the system. Screen 602 is where the view of the operation of system 608 are displayed. View 604 is produced through the use of a schematic of the enclosed space 610, with a dot representing system 608 within a pre-established layout. One of ordinary skill in the art would realize that the schematic could be preloaded in either mobile device 600 or system 608 for display at a later time. Conversely, mobile device 600 could display, as view 606 shown in the other depiction on screen 602, the same enclosed space 610, as an image produced in real or near-real time from a camera or other sensor within enclosed space 610.

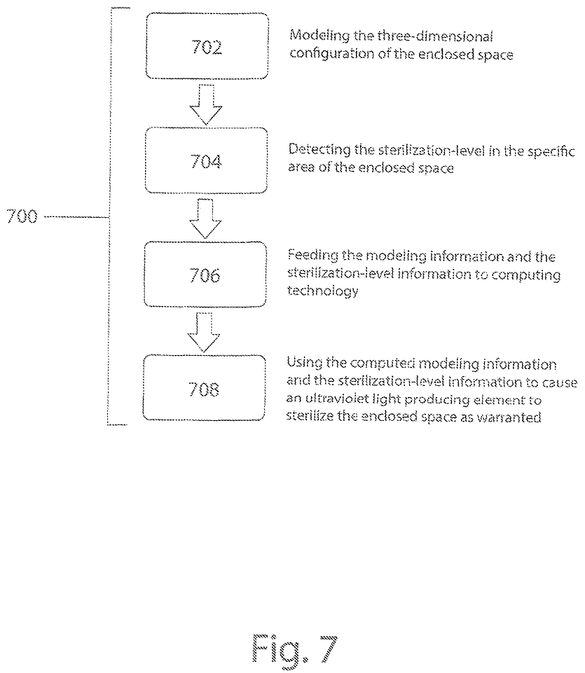

As depicted in FIG. 7, there is also a process version of the present invention—700. The inventive method, a process through which an enclosed space can be sterilized, with the sterilization monitored, includes the steps of (A) modeling the three-dimensional configuration of the enclosed space—702; (B) detecting the sterilization-level in the specific areas of the enclosed space—704; (C) feeding the modeling information and the sterilization-level information to computing technology—706; and (D) using the computed modeling information and the sterilization-level information to cause an ultraviolet light producing element to sterilize the enclosed space as warranted—708. It would be the computed information that would dictate the production of the ultraviolet light. Further, the system would be positioned in a fashion that maximizes the ultraviolet illumination to disinfect the enclosed space to the maximum degree allowable based upon the levels and areas of sterilization detected as necessary.

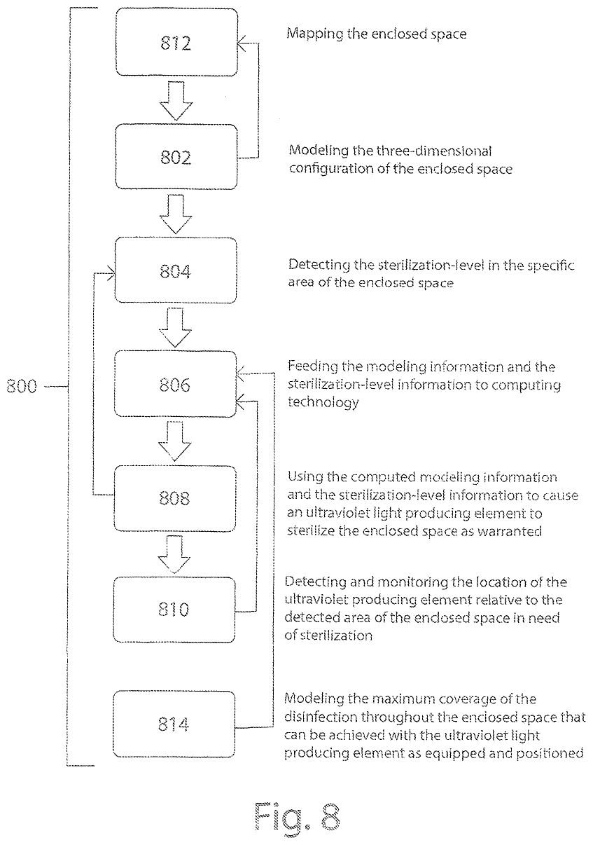

In a further embodiment of the present invention—800, as seen in FIG. 8, the method comprises an additional step of detecting and monitoring the location of the ultraviolet producing element relative to the detected areas of the enclosed space in need of the warranted levels of sterilization—810. Additionally, the inventive method could comprise the step of mapping the enclosed space—812. Such mapping could enable the sterilization to be performed with reference to information available to the ultraviolet producing element, and could be usable in the computing step with regards to obstacles in and other physical factors about the enclosed area. As a result, the sterilization can be performed in a fashion to maximize the ultraviolet illumination in disinfecting the enclosed with reference to the mapped obstacles and other physical factors. The sterilization-level detecting step could also collect the detail the computing steps need to model the maximum coverage of the disinfection throughout the enclosed space that can be achieved with the ultraviolet light producing element as equipped and positioned—814. Moreover, the computing step could use the model to dictate the placement of ultraviolet light producing element throughout the disinfecting process. The mapping could also be in three dimensions, with the positioning of the ultraviolent producing element during the ultraviolent light producing step similarly in three dimensions.

In a different embodiment of the present method—900, it comprises the step of storing data—918 about (A) the positioning of the ultraviolet light producing element (positioning via step 916), (B) the levels of disinfecting within the specific areas of the enclosed space (with detecting and monitoring via step 910), and (C) other pertinent information within the system. Alternatively or additionally, the present inventive method comprises the step of communicating data about the positioning of the ultraviolet light producing element, the levels of disinfecting within the specific areas of the enclosed space, and other pertinent information, to a remote device. One of ordinary skill in the art would realize that the device may have to be in more than one position to ensure full coverage of the enclosed space, and that the device may be manually positioned or self-mobile.

In another preferred embodiment, the inventive method includes the step of visualizing of the location of the ultraviolet light producing element relative to obstacles and other physical factors. An added step could be logging the time and date along with the duration that the ultraviolet light producing element was at each location and operating. Further, the inventive method could comprise the step of creating of a three-dimensional coverage map of all areas disinfected and not disinfected by the ultraviolet light producing element while at each location.

Still other embodiments of the present inventive method could include combinations of steps like, for example, the following:

### Example A

Modeling the 3-D configuration of the enclosed space;
Mapping the enclosed space, thus enabling the sterilization to be performed with respect to information about obstacles and other physical factors to maximize UV illumination in the enclosed space;
Feeding the modeling information and the map to computing technology;
Computing technology estimates the amount of UV light needed to sterilize the enclosed space;
Positioning the system to maximize UV illumination;
Producing UV as directed by the computing technology;
Monitoring the system location relative to areas that needs sterilization; and
Re-positioning the system as directed by the computing technology to maximize UV illumination to all surfaces;

### Example B

Feeding information about an enclosed space to the computing technology;
Estimating through the use of the computing technology the amount of UV light needed to sterilize the enclosed space;
Determining sterilization-level detector in the sections of the enclosed space being sterilized;
Positioning the system in an attempt to maximize UV illumination;
Producing UV based upon the information available to the computing technology;
Monitoring the sterilization level;
Evaluating the sterilization level achieved; and
Re-positioning and producing more UV as needed

### Example C

Estimating through computing technology the amount of UV light needed to sterilize an enclosed space;
Modeling the 3-D configuration of the enclosed space using the estimates and other information;
Determining the position for the system relative to areas that need sterilization based upon the estimates;
Position the system as determined for maximum UV illumination; and
Producing UV as directed by the computing technology to sterilize the enclosed space

### Example D

Modeling the 3-D configuration of an enclosed space, enabling the sterilization to be performed with respect to information about obstacles and other physical factors to maximize UV illumination in the enclosed space using camera and/or other sensors strategically located within the enclosed space;
Feeding the modeling information to the computing technology;
Estimating through the use of the computing technology the amount of UV light needed to sterilize the enclosed space;
Positioning the system, through the use of its own propulsion system, to maximize UV illumination;
Producing UV as directed by the computing technology to sterilize the enclosed space; and
Monitoring the system location relative to areas that need sterilization

The order of the steps in the foregoing examples should not be construed as the only order possible and one or ordinary skill in the art would realize there could be additional steps for each embodiment and loops/iterations between the designated steps.

### ADDITIONAL THOUGHTS

The foregoing descriptions of the present invention have been provided for the purposes of illustration and description. It is not intended to be exhaustive or to limit the invention to the precise forms disclosed. Many modifications and variations will be apparent to the practitioner of ordinary skilled in the art. Particularly, it would be evident that while the examples described herein illustrate how the inventive system may look and how the inventive process may be performed. Further, other elements and/or steps may be used for and provide benefits to the present invention. The depictions of the present invention as shown in the exhibits are provided for purposes of illustration.

The embodiments were chosen and described in order to best explain the principles of the invention and its practical application, thereby enabling others of ordinary skill in the art to understand the invention for various embodiments and with various modifications that are suited to the particular use contemplated.

1. A system for monitoring and sterilizing an enclosed space, comprising:
a housing;
a power supply situated within the housing;
a sterilization-level detector, situated within the housing and connected to the power supply, with the sterilization-level detecting element exposed to the outside of the housing;
a location detector for detecting the position of the system within the enclosed space, with such location detector connected to the power supply and situated within the housing; and
computing technology situated within the housing and connected to the power supply, wherein the computing technology monitors the location of the system through the information supplied by the location detector, and based in part upon the location information feed to the computing technology by the location detector, and the information feed to the computing technology by the sterilization-level detector, which is also electronically connected to the computing technology, as warranted, sends a signal to a ultraviolet light producing element within the enclosed space to maximize the ultraviolet illumination disinfecting the enclosed space to the maximum degree allowable based upon the levels within and areas of the enclosed space detected as in need of being sterilized.

2. The system of claim 1 further comprising a propulsion element attached to the housing and connected to the power supply and the computing technology, wherein the system can move based in part upon the information received by the computing technology to facilitate the maximum levels and areas sterilized.

3. The system of claim 1 wherein the ultraviolet light producing element is situated within the housing and is connected to the power supply, with the ultraviolet illuminating element exposed to the outside of the housing.

4. The system of claim 1 further comprising a propulsion element attached to the housing and connected to the power supply and the computing technology, wherein the system can move based in part upon the information received by the computing technology to facilitate the maximum levels and areas sterilized and wherein the ultraviolet light producing element is situated within the housing and is connected to the power supply, with the ultraviolet illuminating element exposed to the outside of the housing.

5. The system of claim 4 further comprising mapping technology within the housing and exposed to the outside surface of the housing, and connected to the power supply and the computing technology, wherein the mapping technology enables the computing technology to receive information about obstacles and other physical factors about the enclosed space, such that the system can be moved to maximize the ultraviolet illumination in disinfecting the enclosed space with reference to the mapped obstacles and other physical factors.

6. The system of claim 1 wherein sterilization-level detector provides the detail the computing technology needs to model the maximum coverage of the disinfection throughout the enclosed space that can be achieved with the ultraviolet light producing element as equipped and positioned.

7. The system of claim 4 wherein the computing technology uses a model of the enclosed space to initiate and control the movement of the system throughout the disinfecting process.

8. The system of claim 7 wherein the mapping is in three dimensions and the movement of the ultraviolent light producing element during illumination is in three dimensions.

9. The system of claim 1 further comprising storage, situated within the housing and connected to the power supply and the computing technology, for data about the positioning of the system, the levels of disinfecting within the specific areas of the enclosed space, and other pertinent information.

10. The system of claim 1 further comprising a communication element, situated within the housing and connected to the power supply and the computing technology, through which data about the positioning of the system, the levels of disinfecting within the specific areas of the enclosed space, and other pertinent information, can be transmitted to a remote device.

11. The system of claim 1 where data about the relative position of the unit in relation to obstacles detected is displayed.

12. The system of claim 1 where the desired position of the unit in relation to obstacles detected is displayed after the system is the manually placed in the enclosed space.

13. The system of claim 1 where the actual and optimal coverage of the system is communicated such that the relative level of disinfection of all parts of the space can be determined.

14. A method for monitoring and sterilizing an enclosed space, comprising the steps of
modeling the three-dimensional configuration of the enclosed space;
detecting the sterilization-level in the specific areas of the enclosed space;
feeding the modeling information and the sterilization-level information to computing technology; and
using the computed modeling information and the sterilization-level information to cause an ultraviolet light producing element sterilize the enclosed space as warranted, wherein the computed information dictates the production of the ultraviolet light and wherein further the system moves in a fashion that maximizes the ultraviolet illumination to disinfect the enclosed space to the maximum degree allowable based upon the levels and areas of sterilization detected, as necessary.

15. The method of claim 14 further comprising the step of detecting and monitoring the location of the ultraviolet producing element relative to the detected areas of the enclosed space in need of warranted levels of sterilization.

16. The method of claim 14 further comprising the step of mapping the enclosed space, wherein the mapping enables the sterilization to be performed with reference to information received and usable in the computing step about obstacles and other physical factors about the enclosed area, such that the sterilization is performed in a fashion to maximize the ultraviolet illumination in disinfecting the enclosed with reference to the mapped obstacles and other physical factors.

17. The method of claim 14 wherein the sterilization-level detecting step collects the detail the computing steps need to model the maximum coverage of the disinfection throughout the enclosed space that can be achieved with the ultraviolet light producing element as equipped and positioned.

18. The method of claim 14 wherein the computing step uses the model to initiate and control the placement of ultraviolet light producing element throughout the disinfecting process.

19. The method of claim 16 wherein the mapping is in three dimensions and the movement of the ultraviolent producing element during the ultraviolent light producing step is in three dimensions.

20. The method of claim 14 further comprising the step of storing data about the positioning and movement of the ultraviolet light producing element, the levels of disinfecting within the specific areas of the enclosed space, and other pertinent information within the system.

21. The method of claim 14 further comprising the step of communicating data about the positioning and movement of the ultraviolet light producing element, the levels of disinfecting within the specific areas of the enclosed space, and other pertinent information, to a remote device.

22. A system for monitoring and sterilizing an enclosed space, comprising:
a housing;
a power supply situated within the housing;
a sterilization-level detector, situated within the housing and connected to the power supply, with the sterilization-level detecting element exposed to the outside of the housing;
a location detector for detecting the position of the system within the enclosed space, with such location detector connected to the power supply and situated within the housing;
an ultraviolet light producing element is situated within the housing and connected to the power supply, with the ultraviolet illuminating element exposed to the outside of the housing;
computing technology situated within the housing and connected to the power supply, wherein the computing technology monitors the location of the system through the information supplied by the location detector, and based in part upon the location information feed to the computing technology by the location detector, and the information feed to the computing technology by the sterilization-level detector, which is also electronically connected to the computing technology, as warranted, sends a signal to the ultraviolet light producing element to maximize the ultraviolet illumination disinfecting the enclosed space to the maximum degree allowable based upon the levels within and areas of the enclosed space defected as in need of being sterilized;
a propulsion element attached to the housing and connected to the power supply and the computing technology, wherein the system can move based in part upon the information received by the computing technology to facilitate the maximum levels and areas sterilized;
mapping technology within the housing and exposed to the outside surface of the housing, and connected to the power supply and the computing technology, wherein the mapping technology enables the computing technology to receive information about obstacles and other physical factors about the enclosed space, such that the system can be moved to maximize the ultraviolet illumination in disinfecting the enclosed space with reference to the mapped obstacles and other physical factors and wherein sterilization-level detector provides the detail the computing technology needs to model the maximum coverage of the disinfection throughout the enclosed space that can be achieved with the ultraviolet light producing element as equipped and positioned and the computing technology uses a model of the enclosed space to initiate and control the movement of the system throughout the disinfecting process;
storage, situated within the housing and connected to the power supply and the computing technology, for data about the positioning of the system, the levels of disinfecting within the specific areas of the enclosed space, and other pertinent information; and
a communication element, situated within the housing and connected to the power supply and the computing technology, through which data about the positioning of the system, the levels of disinfecting within the specific areas of the enclosed space, and other pertinent information, can be transmitted to a remote device.
